(19) United States
(12) Patent Application Publication
(10) Pub. No.: US 2024/0033500 A1
(43) Pub. Date: Feb. 1, 2024

(54) EVALUATING METHOD FOR USEFUL LIFESPAN OF CONDUCTIVE GEL AND NON-IMPLANTABLE ELECTRICAL STIMULATION DEVICE

(71) Applicant: GIMER MEDICAL. Co. LTD., New Taipei City (TW)

(72) Inventors: Wei-Chih HUANG, New Taipei City (TW); Jian-Hao PAN, New Taipei City (TW); Wan Ting CHIANG, New Taipei City (TW); Chia-Chi WANG, New Taipei City (TW)

(73) Assignee: GIMER MEDICAL. Co. LTD., New Taipei City (TW)

(21) Appl. No.: 18/148,108

(22) Filed: Dec. 29, 2022

(30) Foreign Application Priority Data
Jul. 29, 2022 (CN) 202210909227.2

(51) Int. Cl. A61N 1/04; A61N 1/36

(52) U.S. Cl. CPC A61N 1/0456; A61N 1/36021

(57) ABSTRACT

An evaluating method for a useful lifespan of a conductive gel is applied to a non-implantable electrical stimulation device. The non-implantable electrical stimulation device includes an electrical stimulator and an electrode assembly. The electrical stimulator is detachably electrically connected to the electrode assembly. The evaluating method for the useful lifespan of the conductive gel includes the following steps. A first measuring signal is generated, and the first measuring signal flows through a conductive area to generate a first signal to be tested. The first signal to be tested is received. The first total impedance value is obtained according to the first signal to be tested. The impedance value of the conductive gel is obtained according to the first total impedance value, so as to evaluate the useful lifespan of the conductive gel.

### CROSS REFERENCE TO RELATED APPLICATIONS

This application claims priority of China Patent Application No. 202210909227.2, filed on Jul. 29, 2022, the entirety of which is incorporated by reference herein.

### BACKGROUND

### Technology Field

The disclosure relates to an electrical stimulation device, and, in particular, to an evaluating method for a useful lifespan of a conductive gel and a non-implantable electrical stimulation device.

### Description of the Related Art

In recent years, dozens of therapeutic nerve electrical stimulation devices have been developed, such as non-implantable electrical stimulation devices. A non-implantable electrical stimulation device applies a current to a specific area of the body, so as to suppress pain or relieve other conditions. Commonly, the electrodes of the non-implantable electrical stimulation device are placed on the skin, adjacent or close to the area to be treated (such as the area of pain). There is usually a gel attached to the electrodes, and the gel may contact the skin of the user. The gel has the following functions. One is to transmit the pulse signal evenly to the skin, to avoid uneven contact between the electrode surface and the non-planar skin. Otherwise, the current density of the current on some people's skin to be too great, causing danger (such as burns or pain). Another is to use the viscosity of the gel to allow the non-implantable electrical stimulation device to be attached to the skin.

However, the gel has a finite useful lifespan, and is a consumable with the number of uses. In addition, the user may not necessarily record the number of times that the gel is used. If the recommended number of uses is exceeded, the effect of the treatment may be poor, which may increase the troubles of the user. Therefore, how to effectively determine the useful state of the gel's remaining effectiveness as a basis for replacing the gel and increasing the convenience and safety of the user has become an important issue.

### SUMMARY

The disclosure provides an evaluating method for a useful lifespan of a conductive gel and a non-implantable electrical stimulation device, thereby effectively obtaining the impedance value of the conductive gel to determine the useful lifespan of the conductive gel, so as to increase the convenience of use.

An embodiment of the present disclosure provides an evaluating method for a useful lifespan of a conductive gel, which is applied to a non-implantable electrical stimulation device. The non-implantable electrical stimulation device includes an electrical stimulator and an electrode assembly. The electrical stimulator is detachably electrically connected to the electrode assembly. The evaluating method for the useful lifespan of the conductive gel includes the following steps. A first measuring signal is generated, wherein the first measuring signal flows through a conductive area to generate a first signal to be tested. The first signal to be tested is received. The first total impedance value is obtained according to the first signal to be tested. The impedance value of the conductive gel is obtained according to the first total impedance value, so as to determine the useful lifespan of the conductive gel.

An embodiment of the present disclosure provides a non-implantable electrical stimulation device, which includes an electrode assembly and an electrical stimulator. The electrode assembly includes a conductive gel. The electrical stimulator is detachably electrically connected to the electrode assembly. The electrical stimulator includes an electrical stimulation signal generating circuit and a control unit. The electrical stimulation signal generating circuit is configured to generate a first measuring signal, and the first measuring signal flows through a conductive area to generate a first signal to be tested. The control unit is configured to receive the first signal to be tested, obtain the first total impedance value according to the first signal to be tested, and obtain the impedance value of the conductive gel according to the first total impedance value, so as to evaluate the useful lifespan of the conductive gel.

According to the evaluating method for the useful lifespan of the conductive gel and the non-implantable electrical stimulation device disclosed by the present disclosure, the first measuring signal is generated, and the first measuring signal flows through the conductive area to generate the first signal to be tested. The first total impedance value is obtained according to the first signal to be tested. The impedance value of the conductive gel is obtained according to the first total impedance value, so as to determine the useful lifespan of the conductive gel. Therefore, the impedance value of the conductive gel may be effectively obtained to determine the useful lifespan of the conductive gel, and the convenience of use is increased.

### BRIEF DESCRIPTION OF THE DRAWINGS

The disclosure can be more fully understood by reading the subsequent detailed description and examples with references made to the accompanying drawings, wherein:

FIG. 1A is a perspective view of a non-implantable electrical stimulation device according to an embodiment of the disclosure;

FIG. 1B is a perspective view of a non-implantable electrical stimulation device shown in FIG. 1A form another angle;

FIG. 1C is an exploded schematic view of a non-implantable electrical stimulation device shown in FIG. 1A;

FIG. 2 is a block diagram of a non-implantable electrical stimulation device according to an embodiment of the disclosure;

FIG. 3 is a waveform diagram of an electrical stimulation signal of a non-implantable electrical stimulation device according to an embodiment of the disclosure;

FIG. 4 is an exploded schematic view of a non-implantable electrical stimulation device according to an embodiment of the disclosure;

FIG. 5 is a schematic cross-sectional view of a portion of an electrode assembly according to an embodiment of the disclosure;

FIG. 6A is a perspective view of a non-implantable electrical stimulation device and a storage box according to an embodiment of the disclosure;

FIG. 6B is a perspective view of a non-implantable electrical stimulation device and a storage box according to an embodiment of the disclosure;

FIG. 7 is a perspective view of a non-implantable electrical stimulation device and a storage box according to an embodiment of the disclosure;

FIG. 8 is a flowchart of an evaluating method for a useful lifespan of a conductive gel according to an embodiment of the disclosure;

FIG. 9 is a flowchart of an evaluating method for a useful lifespan of a conductive gel according to an embodiment of the disclosure;

FIG. 10 is a flowchart of an evaluating method for a useful lifespan of conductive gel according to an embodiment of the disclosure;

FIG. 11 is a flowchart of an evaluating method for a useful lifespan of a conductive gel according to an embodiment of the disclosure; and

FIG. 12 is a flowchart following step S808 of FIG. 8, step S908 of FIG. 9, step S1008 of FIG. 10 and step S1108 of FIG. 11.

### DETAILED DESCRIPTION OF THE DISCLOSURE

Technical terms of the present disclosure are based on general definition in the technical field of the present disclosure. If the present disclosure describes or explains one or some terms, definition of the terms is based on the description or explanation of the present disclosure. Each of the disclosed embodiments has one or more technical features. In possible implementation, a person skilled in the art would selectively implement all or some technical features of any embodiment of the present disclosure or selectively combine all or some technical features of the embodiments of the present disclosure.

In each of the following embodiments, the same reference number represents the same or a similar element or component.

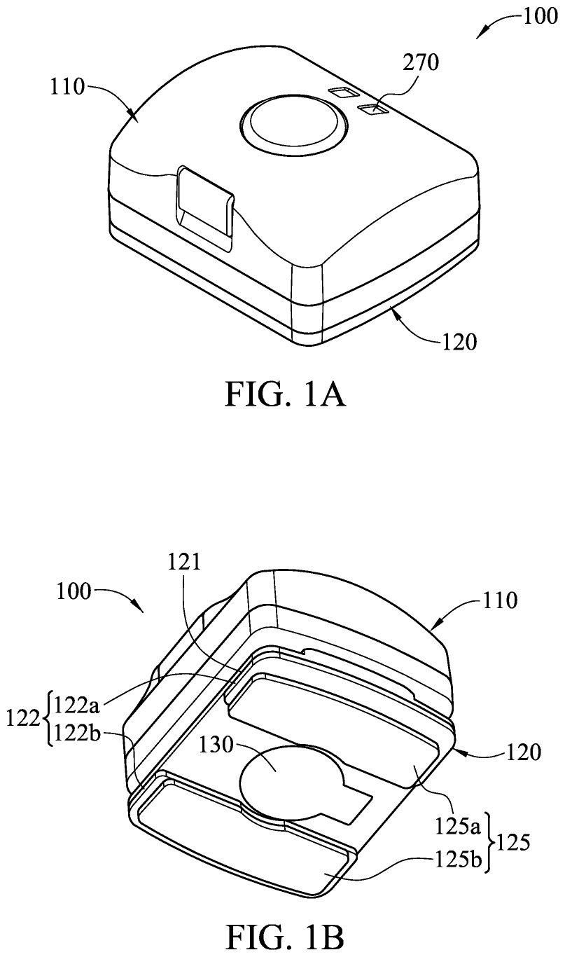

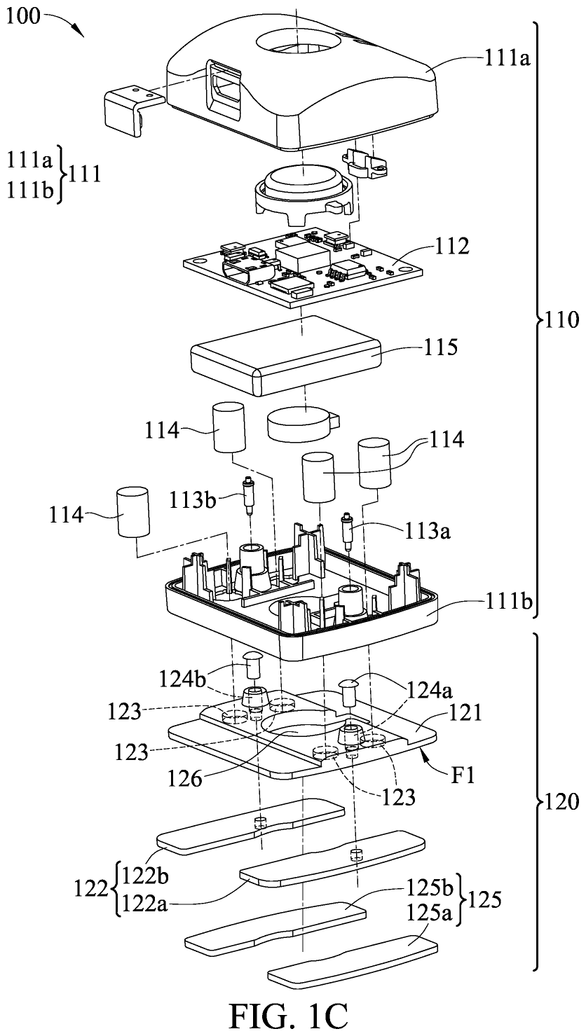

FIG. 1A is a perspective view of a non-implantable electrical stimulation device according to an embodiment of the disclosure. FIG. 1B is a perspective view of a non-implantable electrical stimulation device shown in FIG. 1A form another angle. FIG. 1C is an exploded schematic view of a non-implantable electrical stimulation device shown in FIG. 1A. Please refer to FIG. 1A, FIG. 1B and FIG. 1C. The non-implantable electrical stimulation device 100 includes an electrical stimulator 110 and an electrode assembly 120. In the embodiment, the non-implantable electrical stimulation device 100 is, for example, a transcutaneous electrical nerve stimulation device (TENS device), which does not necessarily need to be implanted in the body or subcutaneously, but is directly attached to the body surface or skin of a living body through the electrode assembly 120 for electrical stimulation of an area to be treated.

In some embodiments, the living body is, for example, the body of a user or a patient. The area to be treated includes, for example, the body surface or skin of the living body, and the area to be treated is, for example, a superficial nerve within 10 millimeters (mm) from the body surface to relieve pain or other symptoms of disease. In addition, the main difference between the non-implantable electrical stimulation device 100 of the embodiment and the general muscle electrical stimulation device is that the area to be treated for electrical stimulation performed by the non-implantable electrical stimulation device 100 of the embodiment is the nerve, rather than the muscle.

In the embodiment, the electrical stimulator 110 is disposed on the upper half of the non-implantable electrical stimulation device 100. The electrical stimulator 110 includes a casing 111, a circuit board 112, two first electrical connectors 113a and 113b and at least one first magnetic unit 114.

The casing 111 includes an upper casing 111a and a lower casing 111b. The upper casing 111a and the lower casing 111b are combined to form an accommodating space. Most of the components required for the electrical stimulator 110 are disposed in the accommodating space, including the circuit board 112, the first electrical connectors 113a and 113b, the first magnetic unit 114, and other components. In the embodiment, the first electrical connectors 113a and 113b are, for example, pogo pins.

On the other hand, the electrode assembly 120 is disposed in the lower half of the non-implantable electrical stimulation device 100 and is connected with the lower casing 111b under of the electrical stimulator 110. The electrode assembly 120 includes a body 121, a conductive component 122 (a first sub conductive component 122a and a second sub conductive component 122b), at least one second magnetic unit 123, two second electrical connectors 124a and 124b and a conductive gel 125 (a first sub conductive gel 125a and a second sub conductive gel 125b). The second electrical connectors 124a and 124b may be separately connected to the first electrical connectors 113a and 113b.

In the embodiment, the second electrical connectors 124a and 124b are, for example, rivets or metal buttons, but the embodiment of the disclosure is not limited thereto. The electrical stimulator 110 may electrically transmit the sent electrical stimulation signal (or the measuring signal) from the circuit board 112 to other components (such as the first electrical connectors 113a and 113b, the second electrical connectors 124a and 124b, the conductive component 122 (the first sub conductive component 122a and the second sub conductive component 122b) and the conductive gel 125 (the first sub conductive gel 125a and the second sub conductive gel 125b)), such that the non-implantable electrical stimulation device 100 may perform electrical stimulation on the area to be treated of the living body.

In the embodiment, the body 121 of the electrode assembly 120 has certain flexibility, such that it may be easily attached to different parts of the living body, and the material of the body 121 of the electrode assembly 120 may be rubber, silicone or other flexible materials.

In the embodiment, the electrode assembly 120 may be a magnetic electrode assembly. In addition, the above conductive component 122 may be a carbon film or another suitable component, but the embodiment of the disclosure is not limited thereto. Furthermore, the above conductive component 122 is attached to a surface F1 of the body 121 opposite to the casing 111. The surface F1 is the lower surface of the body 121 shown in FIG. 1C, and is also a side facing the using part of the user during use. Moreover, an isolation component (not shown) is further disposed on the surface F1 of the body 121, and the isolation component is, for example, a non-woven fabric, but the embodiment of the disclosure is not limited thereto. That is, the above conductive component 122 is attached to the isolation component. In the embodiment, the purpose of the isolation component is not only an insulating effect, but also an auxiliary effect of connecting the conductive component 122 and the body 121, so as to avoid the situation that the adhesion effect may be poor if the conductive component 122 is directly attached to the body 121.

The above second electrical connectors 124a and 124b may, for example, penetrate through the conductive component 122 and be electrically connected to the conductive gel 125. In some embodiments, when using the non-implantable electrical stimulation device 100 of the embodiment, the conductive gel 125 of the electrode assembly 120 may be attached to the above conductive component 122. The conductive gel 125 is not only sticky and may be attached to the body surface or skin of the living body, but also may make the current of the conductive gel 125 evenly spread over the entire attached body surface area, avoiding the stinging sensation of the living body. At the same time, the comfort of using the non-implantable electrical stimulation device 100 is increased. That is, the electrode assembly 120 of the embodiment does not have a lead type, which is different from an implantable stimulation device. The size of the above conductive component 122 may be greater than or equal to the size of the conductive gel 125.

Furthermore, the first magnetic unit 114 of the electrical stimulator 110 is disposed in the accommodating space, for example, between the circuit board 112 and the casing 111. It should be noted that the first magnetic unit 114 in the embodiment is disposed under the circuit board 112.

In the non-implantable electrical stimulation device 100 of the embodiment, the electrical stimulator 110 includes at least one first magnetic unit 114, the electrode assembly 120 includes at least one second magnetic unit 123, and the numbers of the first magnetic unit 114 and the second magnetic unit 123 may be the same or different. The embodiment is described by taking as an example that four first magnetic units 114 correspond to four second magnetic units 123. In addition, the electrode assembly 120 is detachably positioned on one side of the electrical stimulator 110 (i.e., one side of the lower casing 111b of the electrical stimulator 110) by being adsorbed by the at least one first magnetic unit 114 and the at least one second magnetic unit 123.

In addition, in the embodiment, the lower casing 111b of the electrical stimulator 110 may be correspondingly designed to have a protruding configuration 130 (as shown in FIG. 1B) at a position corresponding to the opening 126 of the body 121. After the electrode assembly 120 is assembled to the electrical stimulator 110, the protruding configuration 130 of the lower casing 111b protrudes from the opening 126 of the body 121. Therefore, the electrode assembly 120 may be more stably disposed on the electrical stimulator 110, and the alignment of the electrode assembly 120 and the electrical stimulator 110 is facilitated.

After the electrical stimulation signal is sent from the circuit board 112, the electrical stimulator 110 may be electrically connected to conductive gel 125 through the first electrical connectors 113a and 113b, the second electrical connectors 124a and 124b and the conductive component 122 in sequence, and finally the electrical stimulation signal electrically stimulates the area to be treated through the conductive gel 125. In the embodiment, the area to be treated is a conductive area. In the embodiment, in addition to above components, the non-implantable electrical stimulation device 100 is provided with a battery 115 or a power module in the accommodating space of the electrical stimulator 110, and the battery 115 or the power module may output power to the circuit board 112. It should be noted that the conductive gel 125 may also be replaced with another suitable material, such as an adhesive conductive fabric, which may achieve the same effect.

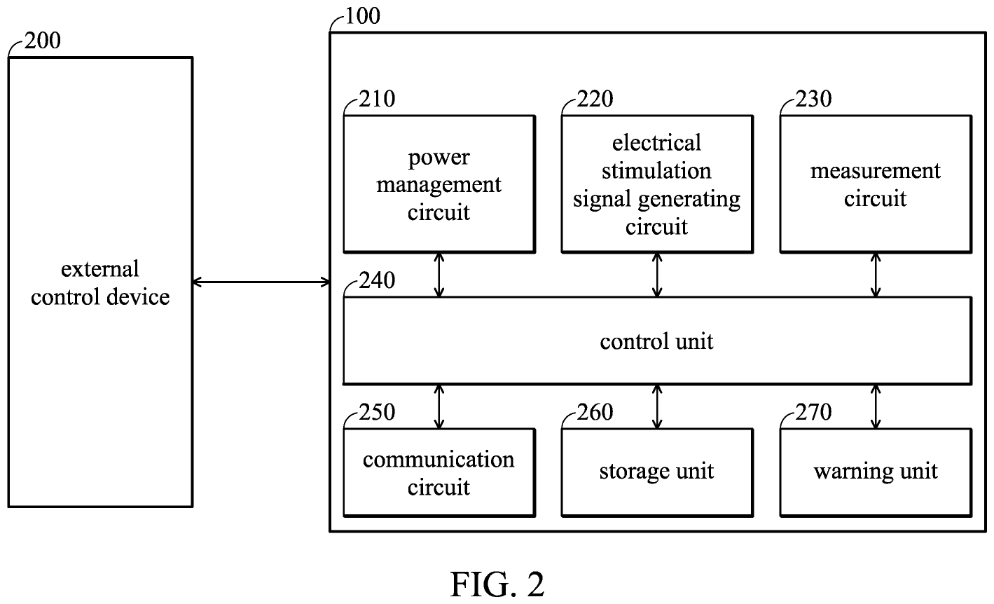

FIG. 2 is a block diagram of a non-implantable electrical stimulation device according to an embodiment of the disclosure. Please refer to FIG. 2. The non-implantable electrical stimulation device 100 may at least include a power management circuit 210, an electrical stimulation signal generating circuit 220, a measurement circuit 230, a control unit 240, a communication circuit 250, a storage unit 260 and a warning unit 270. In addition, the electrical stimulation signal generating circuit 220, the measurement circuit 230, the control unit 240, the communication circuit 250 and the storage unit 260 may be disposed on the circuit board 112 of the electrical stimulator 110 shown in FIG. 1C. Furthermore, the warning unit 270 may be disposed on the casing 111 of the electrical stimulator 110 shown in FIG. 1A and exposed outside the casing 111. It should be noted that the block diagram shown in FIG. 2 is only for the convenience of explaining the embodiment of the disclosure, but the disclosure is not limited to FIG. 2. The non-implantable electrical stimulation device 100 may also include other components.

In some embodiments, the non-implantable electrical stimulation device 100 may be electrically coupled to an external control device 200. The external control device 200 may include an operation interface. According to the operation of the user on the operation interface, the external control device 200 may generate a command or a signal to be transmitted to the non-implantable electrical stimulation device 100, and transmit the command or the signal to the non-implantable electrical stimulation device 100 through a manner of a wired communication (i.e., a transmission line). In some embodiments, the external control device 200 may be a smart phone, but the disclosure is not limited thereto.

Furthermore, in some embodiments, the external control device 200 may also transmit the command or the signal to the non-implantable electrical stimulation device 100 through a manner of a wireless communication (i.e., Bluetooth, Wi-Fi, or near field communication (NFC), but the disclosure is not limited thereto).

In some embodiments, the non-implantable electrical stimulation device 100 and the external control device 200 may be integrated into one device. In some embodiments, the non-implantable electrical stimulation device 100 may be an electrical stimulation device with the battery 115, or an electrical stimulation device with the power wirelessly transmitted by the external control device 200.

In some embodiments, the power management circuit 210 is configured to provide the power to the internal components and circuits in the non-implantable electrical stimulation device 100. The power provided by the power management circuit 210 may be from a built-in rechargeable battery (i.e., the battery 115) or the external control device 200, but the disclosure is not limited thereto. In addition, the external control device 200 may provide the power to the power management circuit 210 through a wireless power supply technology. The power management circuit 210 may be activated or deactivated according to the command of the external control device 200. In some embodiments, the power management circuit 210 may include a switch circuit (not shown). The switch circuit may be turned on or off according to the command of the external control device 200 to activate or deactivate the power management circuit 210.

In some embodiments, the electrical stimulation signal generating circuit 220 is configured to generate the electrical stimulation signal (or the measuring signal). The electrical stimulation signal generating circuit 220 may transmit the generated electrical stimulation signal to the conductive gel 125 of the electrode assembly 122 through the first electrical connectors 113a and 113b, the second electrical connectors 124a and 124b and the conductive component 122, so as to electrically stimulate the area to be treated of the living body (i.e., a human or an animal) through the conductive gel 125. The above area to be treated is, for example, a median nerve, a tibial nerve, a vagus nerve, a trigeminal nerve or other superficial nerves, but the disclosure is not limited thereto.

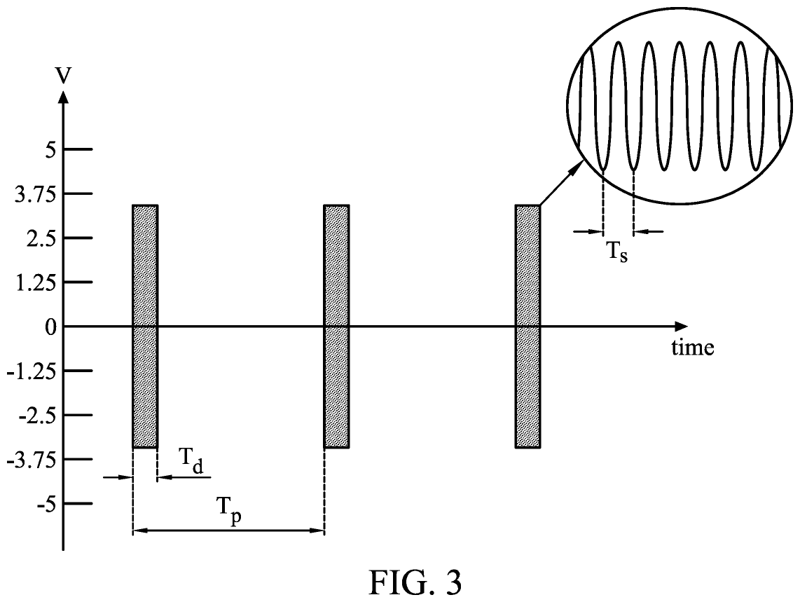

FIG. 3 is a waveform diagram of an electrical stimulation signal of a non-implantable electrical stimulation device according to an embodiment of the disclosure. Please refer to FIG. 3. In some embodiments, the above electrical stimulation signal (or the measuring signal) may be a pulsed radio-frequency (PRF) signal (or referred to as a pulse signal), a continuous sine wave, a continuous triangular wave, a continuous square wave, etc., but the embodiment of the disclosure is not limited thereto. The carrier waveform of the electrical stimulation signal (or the measuring signal) is, for example, a symmetrical biphasic sine wave, but the embodiment of the disclosure is not limited thereto.

In addition, when the electrical stimulation signal (or the measuring signal) is a pulse alternating signal, one pulse cycle time Tp includes one pulse signal and at least one rest period of time, and the pulse cycle time Tp is the reciprocal of the pulse repetition frequency. The pulse repetition frequency range (also referred to as the pulse frequency range) is, for example, between 0 and 1 KHz, preferably between 1 and 100 Hz. In the embodiment, the pulse repetition frequency of the electrical stimulation signal (or the measuring signal) is, for example, 2 Hz.

Furthermore, the duration time T_{d} of the pulse in one pulse cycle time is, for example, between 1 and 250 milliseconds (ms), preferably between 10 and 100 ms. In the embodiment, the duration time T_{d} is, for example, 25 ms. In the embodiment, the frequency of the electrical stimulation signal is 500 KHz, in other words, the cycle time Tₛ of the electrical stimulation signal (or the measuring signal) is about 2 microseconds (s).

Furthermore, the frequency of the above electrical stimulation signal (or the measuring signal) is the intra-pulse frequency in each pulse alternating signal of FIG. 3. In some embodiments, the intra-pulse frequency range of the above electrical stimulation signal is, for example, 1 KHz to 1000 KHz. It should be noted that in each of the embodiments of the disclosure, if only the frequency of the electrical stimulation signal is described, it refers to the intra-pulse frequency of the electrical stimulation signal. In some embodiments, the intra-pulse frequency range of the above electrical stimulation signal is, for example, from 200 KHz to 800 KHz. In some embodiments, the intra-pulse frequency range of the above electrical stimulation signal is, for example, from 480 KHz to 520 KHz. In some embodiments, the intra-pulse frequency of the above electrical stimulation signal is, for example, 500 KHz.

The voltage range of the above electrical stimulation signal (or the measuring signal) may be between −25 V and +25 V. In some embodiments, the voltage range of the above electrical stimulation signal (or the measuring signal) may further be between −10V and +10V. In some embodiments, the voltage range of the above electrical stimulation signal (or the measuring signal) may further be between −4.5V and +4.5V. In addition, the current range of the above electrical stimulation signal (the measuring signal) may be between 0 and 60 mA. In some embodiments, the current range of the above electrical stimulation signal (or the measuring signal) may further be between 0 and 15 mA. In some embodiments, the current range of the electrical stimulation signal (or the measuring signal) is, for example, 8.5 mA.

In some embodiments, the user may operate the non-implantable electrical stimulation device 100 for electrical stimulation only when the user fells the need (for example, the symptoms become severe or not relieved). After the non-implantable electrical stimulation device 100 performs one electrical stimulation on the area to be treated, the non-implantable electrical stimulation device 100 needs to wait a limited time before performing the next electrical stimulation on the area to be treated. For example, after the non-implantable electrical stimulation device 100 performs one electrical stimulation on the area to be treated, the non-implantable electrical stimulation device 100 needs to wait for 30 minutes (i.e., the limited time) before performing the next electrical stimulation on the area to be treated, but the disclosure is not limited thereto. The limited time may also be any time interval within 45 minutes, 1 hour, 4 hours or 24 hours.

In some embodiments, the measurement circuit 230 may measure the voltage value and the current value of the electrical stimulation signal (or the measuring signal) according to the electrical stimulation signal (or the measuring signal) generated by the electrical stimulation signal generating circuit 220. Furthermore, the measurement circuit 230 may measure the voltage value and the current value on the tissue of the area to be treated of the living body (i.e., the body of the user or patient). In some embodiments, the measurement circuit 230 may adjust the current and the voltage of the electrical stimulation signal (or the measuring signal) according to the instruction of the control unit 240.

In some embodiments, the control unit 240 may be a controller, a microcontroller or a processor, but the disclosure is not limited thereto. The control unit 240 may be configured to control the electrical stimulation signal generating circuit 220 and the measurement circuit 230.

In some embodiments, the communication circuit 250 may be configured to communicate with the external control device 200. The communication circuit 250 may transmit the command or the signal received from the external control device 200 to the control unit 240, and transmit the data measured by the non-implantable electrical stimulation device 100 to the external control device 200. In some embodiments, the communication circuit 250 may communicate with the external control device 200 in a wireless or a wired communication manner.

Compared with the traditional electrical stimulation signal, which is a pulse signal with a low frequency (i.e., 10 KHz), it is easy to cause the tingling sensation of the user or discomfort of the user due the paresthesia. In some embodiments, the electrical stimulation signal (or the measuring signal) is a pulse signal with a high frequency (i.e., 500 KHz). Therefore, no paresthesia, or only a very slight paresthesia, may be caused.

In some embodiments, the storage unit 260 may be a volatile memory (i.e., random access memory (RAM)), a non-volatile memory (i.e., flash memory), a read only memory (ROM), a hard disk or a combination thereof. The storage unit 260 may be configured to store files and data required for electrical stimulation. In some embodiments, the storage unit 260 may be configured to store the relevant information of a look-up table provided by the external control device 200.

In addition, the storage unit 260 stores the impedance value of the electrical stimulator 110, the impedance value of the electrode assembly 120 excluding the conductive gel 125 (for example, including the overall impedance of the body 121, the conductive component 122, the second magnetic unit 123 and the two second electrical connectors 124a and 124b) and the predetermined tissue impedance value. Furthermore, the impedance value of the above electrical stimulator 110 and the impedance value of the above electrode assembly 120 excluding the conductive gel 125 may be measured in advance (for example, before leaving the factory) and stored in the storage unit 260. Moreover, the predetermined tissue impedance value is, for example, an impedance value of the skin of the user, and may be preset and stored in the storage unit 260.

In some embodiments, the warning unit 270 may include a light-emitting unit and a sound unit. The light-emitting unit is, for example, a light-emitting diode (LED), and the sound unit is, for example, a horn or a buzzer, but the embodiment of the disclosure is not limited thereto. The warning unit 270 may display the above warning signal according to the warning signal generated by the control unit 240.

In an operation of the non-implantable electrical stimulation device 100, firstly, the electrical stimulator 110 and the electrode assembly 120 are placed in the area to be treated (for example, the electrode assembly 120 has been attached to the skin of the user), and the electrical stimulator 110 is turned on but the course of providing electrical stimulation has not yet started. The control unit 240 controls the electrical stimulation signal generating circuit 220 to generate a first measuring signal. The first measuring signal flows through the area to be treated through the first electrical connector 113a, the second electrical connector 124a, the conductive component 122 and the conductive gel 125, so as to generate a first signal to be tested.

Then, the control unit 240 receives the above first signal to be tested, for example, through the conductive gel 125, the conductive component 122, the second electrical connector 124b and the first electrical connector 113b, and the first signal to be tested includes a voltage value and a current value. Afterward, the control unit 240 obtains the first total impedance value according to the above first signal to be tested. Then, the control unit 240 obtains the impedance value of the conductive gel 125 according to the above first total impedance value, so as to determine the useful lifespan of the conductive gel 125.

For example, in some embodiments, after the control unit 240 obtains the first total impedance value, the control unit 240 may deduct the impedance value of the electrical stimulator 110, the impedance value of the electrode assembly 120 excluding the conductive gel 125 and the predetermined tissue impedance value from the first total impedance, so as to obtain the impedance value of the conductive gel 125 and determine the useful lifespan of the conductive gel 125. In some embodiments, since the impedance value of the electrical stimulator 110 and the impedance value of the electrode assembly 120 excluding the conductive gel 125 are extremely small (such as about 10-20 ohms), the control unit 240 may ignore the impedance value of the electrical stimulator 110 and the impedance value of the electrode assembly 120 excluding the conductive gel 125. That is, the control unit 240 may deduct the predetermined tissue impedance value from the first total impedance value, so as to obtain the impedance value of the conductive gel 125 and determine the useful lifespan of the conductive gel 125. In the embodiment, the range of the predetermined tissue impedance value is, for example, 200-2000 ohms, but the embodiment of the disclosure is not limited thereto. Furthermore, the predetermined tissue impedance value is, for example, 500 ohms.

After the control unit 240 obtains the impedance value of the conductive gel 125, the control unit 240 may generate a warning signal according to the impedance value of the conductive gel 125 and the predetermined impedance value. In the embodiment, the range of the above predetermined impedance value is, for example, 600-800 ohms. For example, the control unit 240 may compare the impedance value of the conductive gel 125 with the predetermined impedance value, so as to determine whether to generate the warning signal. When the impedance value of the conductive gel 125 is smaller than the predetermined impedance value (such as 600-800 ohms), it indicates that the state (the useful lifespan) of the conductive gel 125 is usable, and the control unit 240 may not generate the warning signal. When the impedance value of the conductive gel 125 is larger than or equal to the predetermined impedance value (such as 600-800 ohms), it indicates that the state (the useful lifespan) of the conductive gel 125 is unusable, and the control unit 240 may generate the warning signal. In some embodiments, the predetermined impedance value is set to 600 ohms.

In some embodiments, the warning signal may be transmitted to the warning unit 270, and the warning unit 270 may display the warning signal accordingly, for example, the light-emitting unit emits light or flashes, or the sound unit emits sound. In some embodiments, the warning signal may be transmitted to the external control device 200 through the communication circuit 250, so that the external control device 200 may display the warning signal. Therefore, the user may know the state (the useful lifespan) of the conductive gel 125 through the warning unit 270 or the external control device 200, and replace the conductive gel 125 or the electrode assembly 120, so as to increase the convenience of use.

In the above embodiment, the control unit 240 obtains the impedance value of the conductive gel 125 by using the predetermined tissue impedance value, but the embodiment of the disclosure is not limited thereto. For example, in some embodiments, after the control unit 240 obtains the first total impedance value, the control unit 240 may deduct the impedance value of the electrical stimulator 110 and the impedance value of the electrode assembly 120 excluding the conductive gel 125 from the first total impedance value, so as to obtain the sum of the impedance value of the conductive gel 125 and the tissue impedance value of the skin of the user, and determine the useful lifespan of the conductive gel 125 according to the sum of the impedance value of the conductive gel 125 and the tissue impedance value of the skin of the user.

In some embodiments, since the impedance value of the electrical stimulator 110 and the impedance value of the electrode assembly 120 excluding the conductive gel 125 are extremely small (such as about 10-20 ohms), the control unit 240 may ignore the impedance value of the electrical stimulator 110 and the impedance value of the electrode assembly 120 excluding the conductive gel 125. That is, the first total impedance value is the sum of the impedance value of the conductive gel 125 and the tissue impedance value of the skin of the user.

After the control unit 240 obtains the sum of the impedance value of the conductive gel 125 and the tissue impedance value of the skin of the user, the control unit 240 may compare the sum of the impedance value of the conductive gel 125 and the tissue impedance value of the skin of the user with the predetermined impedance value, so as to determine whether to generate the warning signal. In the embodiment, the range of the above predetermined impedance value is, for example, 1000-2000 ohms, and the preferred predetermined impedance value is 1000 ohms.

For example, when the sum of the impedance value of the conductive gel 125 and the tissue impedance value of the skin of the user is smaller than the predetermined impedance value (such as 1000-2000 ohms), it indicates that the state (the useful lifespan) of the conductive gel 125 is usable, and the control unit 240 may not generate the warning signal. when the sum of the impedance value of the conductive gel 125 and the tissue impedance value of the skin of the user is larger than or equal to the predetermined impedance value (such as 1000-2000 ohms), it indicates that the state (the useful lifespan) of the conductive gel 125 is unusable, and the control unit 240 may generate the warning signal.

In some embodiments, the warning signal may be transmitted to the warning unit 270, and the warning unit 270 may display the warning signal accordingly, for example, the light-emitting unit emits light or flashes, or the sound unit emits sound. In some embodiments, the warning signal may be transmitted to the external control device 200 through the communication circuit 250, so that the external control device 200 may display the warning signal. Therefore, the user may know the state (the useful lifespan) of the conductive gel 125 through the warning unit 270 or the external control device 200, and replace the conductive gel 125 or the electrode assembly 120, so as to increase the convenience of use.

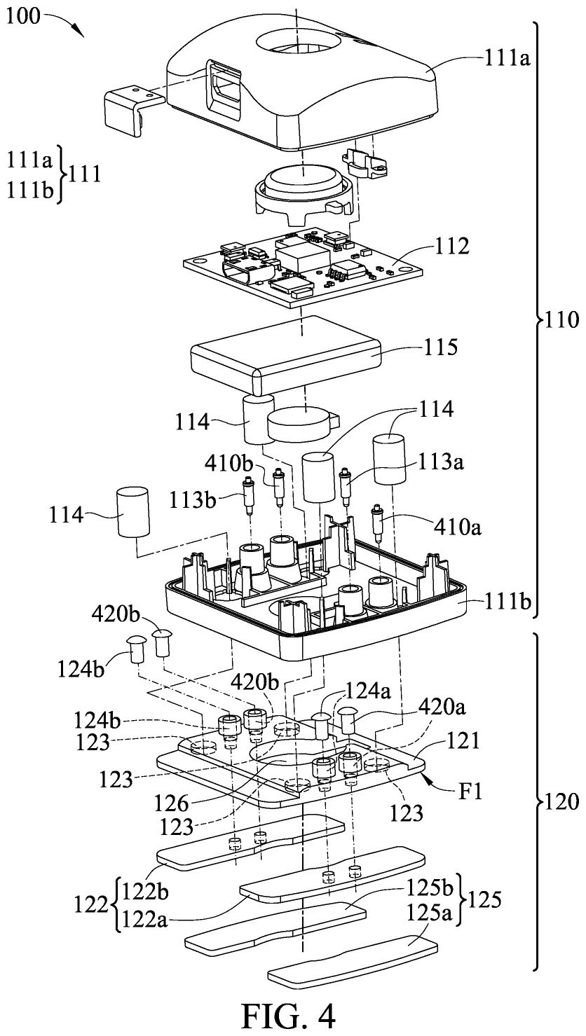

FIG. 4 is an exploded schematic view of a non-implantable electrical stimulation device according to an embodiment of the disclosure. Please refer to FIG. 4. The non-implantable electrical stimulation device 400 includes an electrical stimulator 410 and an electrode assembly 420. The appearances of the electrical stimulator 410 and the electrode assembly 420 in FIG. 4 are the same as or similar to the appearances of the electrical stimulator 110 and the electrode assembly 120 in FIG. 1A and FIG. 1B. Accordingly, the appearances of the electrical stimulator 410 and the electrode assembly 420 in FIG. 4 may refer to FIG. 1A and FIG. 1B, and the description thereof is not repeated herein.

In the embodiment, the electrical stimulator 410 is disposed on the upper half of the non-implantable electrical stimulation device 400. The electrical stimulator 410 includes a casing 111, a circuit board 112, four first electrical connectors 113a, 113b, 410a and 410b and at least one first magnetic unit 114. In addition, the casing 111, the circuit board 112, the first electrical connectors 113a and 113b and the first magnetic unit 114 in FIG. 4 are the same as or similar to the casing 111, the circuit board 112, the first electrical connectors 113a and 113b and the first magnetic unit 114 in FIG. 1C. Accordingly, the casing 111, the circuit board 112, the first electrical connectors 113a and 113b and the first magnetic unit 114 in FIG. 4 may refer to the description of the embodiment of FIG. 1C, and the description thereof is not repeated herein.

In addition, the first electrical connectors 113a and 410a are disposed on the same side, and the first electrical connectors 113b and 410b are disposed on the other side opposite to the first electrical connectors 113a and 410a. In the embodiment, the first electrical connectors 113a, 113b, 410a and 410b are, for example, pogo pins.

On the other hand, the electrode assembly 420 is disposed in the lower half of the non-implantable electrical stimulation device 400 and is connected with the lower casing 111b under of the electrical stimulator 410. The electrode assembly 420 includes a body 121, a conductive component 122, at least one second magnetic unit 123, four second electrical connectors 124a, 124b, 420a and 420b and the conductive gel 125, wherein the conductive component 122 includes a first sub conductive component 122a and a second sub conductive component 122b, and the conductive gel 125 includes a first sub conductive gel 125a and a second sub conductive gel 125b.

In addition, the body 121, the conductive component 122, the second magnetic unit 123, the second electrical connectors 124a and 124b and the conductive gel 125 in FIG. 4 are the same as or similar to the body 121, the conductive component 122, the second magnetic unit 123, the second electrical connectors 124a and 124b and the conductive gel 125 in FIG. 1C. Accordingly, the body 121, the conductive component 122, the second magnetic unit 123, the second electrical connectors 124a and 124b and the conductive gel 125 in FIG. 4 may refer to the description of the embodiment of FIG. 1C, and the description thereof is not repeated herein.

The second electrical connectors 124a, 124b, 420a and 420b may be detachably connected to the first electrical connectors 113a, 113b, 410a and 410b. For example, the second electrical connector 124a is detachably connected to the first electrical connector 113a. The second electrical connector 124b is detachably connected to the first electrical connector 113b. The second electrical connector 420a is detachably connected to the first electrical connector 410a. The second electrical connector 420b is detachably connected to the first electrical connector 410b.

In the embodiment, the first electrical connectors 113a and 410a are disposed between the two first magnetic units 114, the first electrical connectors 113b and 410b are disposed between the two first magnetic units 114, the second electrical connectors 124a and 420a are disposed between the two second magnetic units 123, and the second electrical connectors 124b and 420b are disposed between the two second magnetic units 123, but the embodiment of the disclosure is not limited thereto.

In some embodiments, the positions of the first electrical connectors 113a, 113b, 410a and 410b may be interchanged with the positions of the first magnetic units 114, and the positions of the second electrical connectors 124b, 124b, 420a and 420b may be interchanged with the position of the second magnetic units 114. For example, the two first electrical magnetic units 114 are disposed between the first electrical connectors 113a and 410a, the two first electrical magnetic units 114 are disposed between the first electrical connectors 113b and 410b, the two second electrical magnetic units 123 are disposed between the second electrical connectors 124a and 420a, the two second electrical magnetic units 123 are disposed between the second electrical connectors 124b and 420b, and the same effect may also be achieved.

In the embodiment, the second electrical connectors 124a, 124b, 420a and 420b are, for example, rivets or metal buttons, but the embodiment of the disclosure is not limited thereto. The electrical stimulator 410 may electrically transmit the sent electrical stimulation signal (or the measuring signal) from the circuit board 112 to other components (such as the first electrical connectors 113a, 113b, 410a and 410b, the second electrical connectors 124a, 124b, 420a and 420b, the first sub conductive component 122a, the second sub conductive component 122b, the first sub conductive gel 125a and the second sub conductive gel 125b), such that the non-implantable electrical stimulation device 400 may perform electrical stimulation on the area to be treated of the living body.

The above second electrical connectors 124a, 124b, 420a and 420b may penetrate through, for example, the first sub conductive component 122a and the second conductive component 122b and be electrically connected to the first sub conductive gel 125a and the second sub conductive gel 125b. In the embodiment, the second electrical connectors 124a and 420a are electrically connected to the first sub conductive gel 125a, and the second electrical connectors 124b and 420b are electrically connected to the second sub conductive gel 125b.

In some embodiments, when using the non-implantable electrical stimulation device 400 of the embodiment, the first sub conductive gel 125a and the second sub conductive gel 125b of the electrode assembly 420 may be attached to the above first sub conductive component 122a and the above second sub conductive component 122b. The first sub conductive gel 125a and the second sub conductive gel 125b are not only sticky and may be attached to the body surface or skin of the living body, but also may make the currents of the first sub conductive gel 125a and the second sub conductive gel 125b evenly spread over the entire attached body surface area, avoiding the stinging sensation of the living body. At the same time, the comfort of using the non-implantable electrical stimulation device 400 is increased. That is, the electrode assembly 420 of the embodiment does not have a lead type. The sizes of the above first sub conductive component 122a and the above second sub conductive component 122b may be greater than or equal to the sizes of the above first sub conductive gel 125a and the above second sub conductive gel 125b.

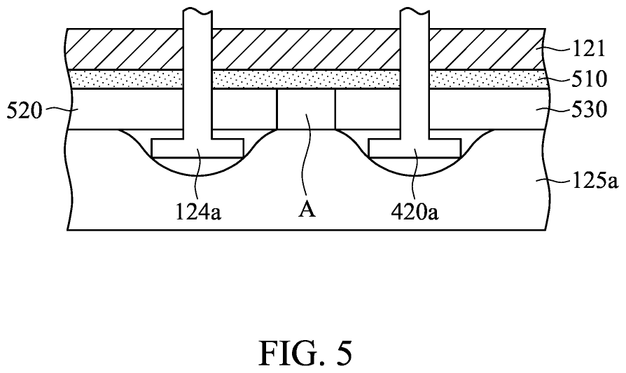

In some embodiments, as shown in FIG. 5, an isolation component 510 (such as a non-woven fabric) may be disposed between the body 121 and the first sub conductive component 122a, and the first sub conductive component 122a may include a first portion 520 and a second portion 530 that are separated. In FIG. 5, it can be seen that the first portion 520 and the second portion 530 are not in contact at a position A, i.e., there is no conductive component disposed at the position A. Similarly, the arrangement of the second sub conductive component 122b is the same as or similar to the arrangement of the first sub conductive component 122a. Accordingly, the arrangement of the second sub conductive component 122b may refer to the description of FIG. 5, and the description thereof is not repeated herein. Therefore, by using the design of FIG. 5, it may be avoided that when measuring the impedance value of the first sub conductive gel 125a or the second sub conductive gel 125b, the measuring signal directly passes through the first sub conductive component 122a or the second conductive component 122b without passing through the first sub conductive gel 125a or the second sub conductive gel 125b, resulting in inaccurate measurement.

Furthermore, the non-implantable electrical stimulation device 400 may include a power management circuit 210, an electrical stimulation signal generating circuit 220, a measurement circuit 230, a control unit 240, a communication circuit 250, a storage unit 260 and a warning unit 270, as shown in FIG. 2. The connection relationship and the operation thereof of the power management circuit 210, the electrical stimulation signal generating circuit 220, the measurement circuit 230, the control unit 240, the communication circuit 250, the storage unit 260 and the warning unit 270 may refer to the description of the embodiment of FIG. 2, and the description thereof is not repeated herein.

In an operation of the non-implantable electrical stimulation device 400, when the non-implantable electrical stimulation device 400 is turned on and has not been attached to the area to be tested, the control unit 240 controls the electrical stimulation signal generating circuit 220 to generate a second measuring signal. The second measuring signal flows through the first sub conductive gel 125a through the first electrical connector 113a, the second electrical connector 124a, the first sub conductive component 122a (the first portion 520), so as to generate a second signal to be tested.

Then, the control unit 240 receives the above second signal to be tested, for example, through the first sub conductive gel 125a, the first sub conductive component 122a (the second portion 530), the second electrical connector 420a and the first electrical connector 410a, and the second signal to be tested includes a voltage value and a current value. Afterward, the control unit 240 obtains the second total impedance value according to the above second signal to be tested. Then, the control unit 240 obtains the impedance value of the first sub conductive gel 125a according to the above second total impedance value, so as to determine the useful lifespan of the conductive gel 125.

For example, in some embodiments, after the control unit 240 obtains the second total impedance value, the control unit 240 may deduct the impedance value of the electrical stimulator 410 and the impedance value of the electrode assembly 420 excluding the conductive gel 125 from the second total impedance, so as to obtain the impedance value of the first sub conductive gel 125a and determine the useful lifespan of the conductive gel 125. In some embodiments, since the impedance value of the electrical stimulator 410 and the impedance value of the electrode assembly 420 excluding the conductive gel 125 are extremely small (such as about 10-20 ohms), the control unit 240 may ignore the impedance value of the electrical stimulator 410 and the impedance value of the electrode assembly 420 excluding the conductive gel 125. That is, the impedance value of the first sub conductive gel 125a obtained by the control unit 240 is the second total impedance value.

In addition, the control unit 240 controls the electrical stimulation signal generating circuit 220 to generate a third measuring signal. The third measuring signal flows through the second sub conductive gel 125b through the first electrical connector 113b, the second electrical connector 124b, the second sub conductive component 122b (the first portion 520) so as to generate a third signal to be tested.

Then, the control unit 240 receives the above third signal to be tested, for example, through the second sub conductive gel 125b the second sub conductive component 122b (the second portion 530), the second electrical connector 420b and the first electrical connector 410b, and the third signal to be tested includes a voltage value and a current value. Afterward, the control unit 240 obtains the third total impedance value according to the above third signal to be tested. Then, the control unit 240 obtains the impedance value of the second sub conductive gel 125b according to the above third total impedance value, so as to determine the useful lifespan of the conductive gel 125.

For example, in some embodiments, after the control unit 240 obtains the third total impedance value, the control unit 240 may deduct the impedance value of the electrical stimulator 410 and the impedance value of the electrode assembly 420 excluding the conductive gel 125 from the third total impedance, so as to obtain the impedance value of the second sub conductive gel 125b and determine the useful lifespan of the conductive gel 125. In some embodiments, since the impedance value of the electrical stimulator 410 and the impedance value of the electrode assembly 420 excluding the conductive gel 125 are extremely small (such as about 10-20 ohms), the control unit 240 may ignore the impedance value of the electrical stimulator 410 and the impedance value of the electrode assembly 420 excluding the conductive gel 125. That is, the impedance value of the second sub conductive gel 125b obtained by the control unit 240 is the second total impedance value. Therefore, when the non-implantable electrical stimulation device 400 is not attached to the area to be tested, the impedance value of the first sub conductive gel 125a and the impedance value of the second sub conductive gel 125b may be measured and obtained to determine the useful lifespan of the conductive gel 125, so as to increase the convenience of use.

After the control unit 240 obtains the impedance values of the first sub conductive gel 125a and the second sub conductive gel 125b, the control unit 240 may generate a warning signal according to the impedance values of the first sub conductive gel 125a and the second sub conductive gel 125b and the predetermined impedance value. In the embodiment, the range of the above predetermined impedance value is, for example, 600-800 ohms.

For example, the control unit 240 may compare the impedance values of the first sub conductive gel 125a and the second sub conductive gel 125b with the predetermined impedance value, so as to determine whether to generate the warning signal. When the impedance values of the first sub conductive gel 125a and the second sub conductive gel 125b are smaller than the predetermined impedance value (such as 600-800 ohms), it indicates that the states (the useful lifespanss) of the first sub conductive gel 125a and the second sub conductive gel 125b are usable, and the control unit 240 may not generate the warning signal. When the impedance values of the first sub conductive gel 125a and the second sub conductive gel 125b are larger than or equal to the predetermined impedance value (such as 600-800 ohm), it indicates that the states (the useful lifespan s) of the first sub conductive gel 125a and the second sub conductive gel 125b are unusable, and the control unit 240 may generate the warning signal.

In some embodiments, the warning signal may be transmitted to the warning unit 270, and the warning unit 270 may display the warning signal accordingly, for example, the light-emitting unit emits light or flashes, or the sound unit emits sound. In some embodiments, the warning signal may be transmitted to the external control device 200 through the communication circuit 250, so that the external control device 200 may display the warning signal. Therefore, the user may know the states (the useful lifespan) of the first sub conductive gel 125a and the second sub conductive gel 125b through the warning unit 270 or the external control device 200, and replace the conductive gel 125 or the electrode assembly 420, so as to increase the convenience of use.

In addition, the non-implantable electrical stimulation device 400 may also measure the impedance value of the conductive gel 125 in the above non-implantable electrical stimulation phase. The relevant operation of the non-implantable electrical stimulation device 400 may refer to the description of the operation of the above non-implantable electrical stimulation device 100, and the description thereof is not repeated herein.

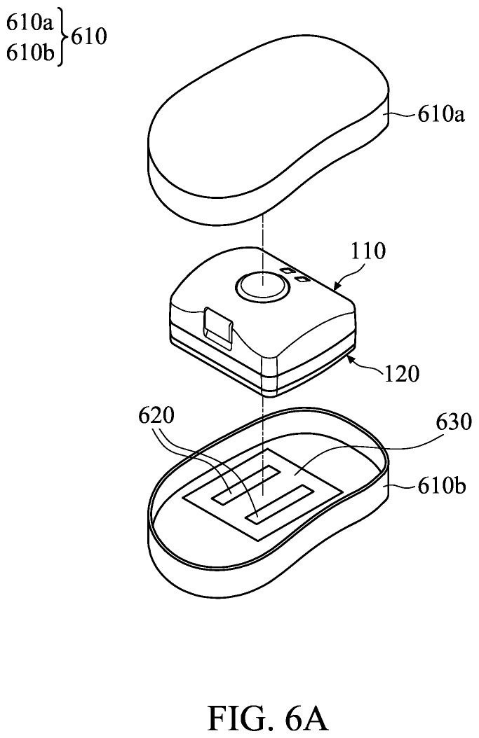

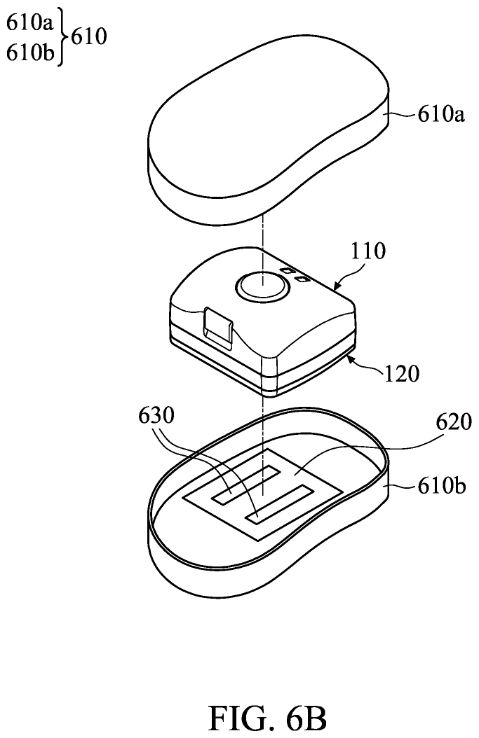

FIG. 6A is a perspective view of a non-implantable electrical stimulation device and a storage box according to an embodiment of the disclosure. Please refer to FIG. 6A. The non-implantable electrical stimulation device 600 includes an electrical stimulator 110, an electrode assembly 120 and a storage box 610. In the embodiment, the electrical stimulator 110 and the electrode assembly 120 in FIG. 6A are the same as or similar to the electrical stimulator 110 and the electrode assembly 120 in FIG. 1A, FIG. 1B, FIG. 1C and FIG. 2. Accordingly, the electrical stimulator 110 and the electrode assembly in FIG. 6A may refer to the description of the embodiment of FIG. 1A, FIG. 1B, FIG. 1C and FIG. 2, and the description thereof is not repeated herein.

In the embodiment, the storage box 610 includes an upper cover 610a and a lower cover 610b. The upper cover 610a and the lower cover 610b form an accommodating space, and the accommodating space is configured to accommodate the electrical stimulator 110 and the electrode assembly 120. In addition, the lower cover 610b may include a conductive component 620 and an isolation component 630, and the conductive component 620 of the lower cover 610b may form an area to be treated (i.e., a conductive area) that simulates the skin of the user. The conductive component 620 is disposed on the lower cover 610b, i.e., the conductive component 620 is disposed on a side of the lower cover 610b adjacent to the electrode assembly 120. The isolation component 630 is disposed on a part of the conductive component 620. That is, the isolation component 630 is attached to the conductive component 620 and exposes the conductive component 620.

In the embodiment, the size of the isolation component 630 is, for example, larger than the size of the conductive component 620, and the part of the conductive component 620 exposed outside the isolation component 630 may correspond to the conductive gel 125, In some embodiments, the size of the isolation component 630 is, for example, smaller than the size of the conductive component 620 and the size of the conductive gel 125, and the position of the isolation component 630 corresponds to the position of the conductive gel 125, as shown in FIG. 6B. In the embodiment, the conductive component 620 is, for example, a carbon film, and the isolation component 630 is, for example, a release paper, but the embodiment of the disclosure is not limited thereto. The above release paper allows the conductive gel 125 to be easily torn.

Furthermore, the storage unit 260 of the electrical stimulator 110 may store the impedance value of the electrical stimulator 110, the impedance value of the electrode assembly 120 excluding the conductive gel 125 and the impedance value of the conductive component 620. In addition, the impedance value of the conductive component 620 may be measured in advance and stored in the storage unit 260.

In an operation of the non-implantable electrical stimulation device 600, the electrical stimulator 110 and the electrode assembly 120 are placed in the conductive area (for example, the electrode assembly 120 has been attached to the conductive component 620 of the lower cover 610b), and the electrical stimulator 110 is turned on but the course of providing electrical stimulation has not yet started. The control unit 240 controls the electrical stimulation signal generating circuit 220 to generate a first measuring signal. The first measuring signal flows through the conductive area (i.e., the conductive component 620) through the first electrical connector 113a, the second electrical connector 124a, the conductive component 122 and the conductive gel 125, so as to generate a first signal to be tested.

Then, the control unit 240 receives the above first signal to be tested, for example, through the conductive gel 125, the conductive component 122, the second electrical connector 124b and the first electrical connector 113b, and the first signal to be tested includes a voltage value and a current value. Afterward, the control unit 240 obtains the first total impedance value according to the above first signal to be tested. Then, the control unit 240 obtains the impedance value of the conductive gel 125 according to the above first total impedance value, so as to determine the useful lifespan of the conductive gel 125.

For example, after the control unit 240 obtains the first total impedance value, the control unit 240 may deduct the impedance value of the electrical stimulator 110, the impedance value of the electrode assembly 120 excluding the conductive gel 125 and the impedance value of the conductive component 620 from the first total impedance, so as to obtain the impedance value of the conductive gel 125 and determine the useful lifespan of the conductive gel 125. Furthermore, since the impedance value of the electrical stimulator 110 and the impedance value of the electrode assembly 120 excluding the conductive gel 125 are extremely small (such as about 10-20 ohms), the control unit 240 may ignore the impedance value of the electrical stimulator 110 and the impedance value of the electrode assembly 120 excluding the conductive gel 125. That is, the control unit 240 may deduct the impedance value of the conductive component 620 from the first total impedance value, so as to obtain the impedance value of the conductive gel 125 and determine the useful lifespan of the conductive gel 125.

Then, the manner in which the control unit 240 generates the warning signal according to the impedance value of the conductive gel 125 and the predetermined impedance value may refer to the description of the above embodiment, and the description thereof is not repeated herein. Therefore, the user may place the non-implantable electrical stimulation device 600 on the lower cover 610b and make the electrode assembly 120 to contact the conductive component 620, so as to perform the operation for measuring the impedance value of the conductive gel 125 as a basis of replacing the conductive gel 125 or the electrode assembly 120 and increase the convenience of use.

In FIG. 6A and FIG. 6B, the non-implantable electrical stimulation device 600 includes the electrical stimulator 110 and the electrode assembly 120, but the embodiment of the disclosure is not limited thereto. In some embodiments, the non-implantable electrical stimulation device 600 may include an electrical stimulator 410 and an electrode assembly 420. The related operations of the electrical stimulator 410 and the electrode assembly 420 are the same as or similar to the related operations of the electrical stimulator 110 and the electrode assembly 120. Accordingly, the related operations of the electrical stimulator 410 and the electrode assembly 420 may refer to the description of the above embodiment, and the description thereof is not repeated herein.

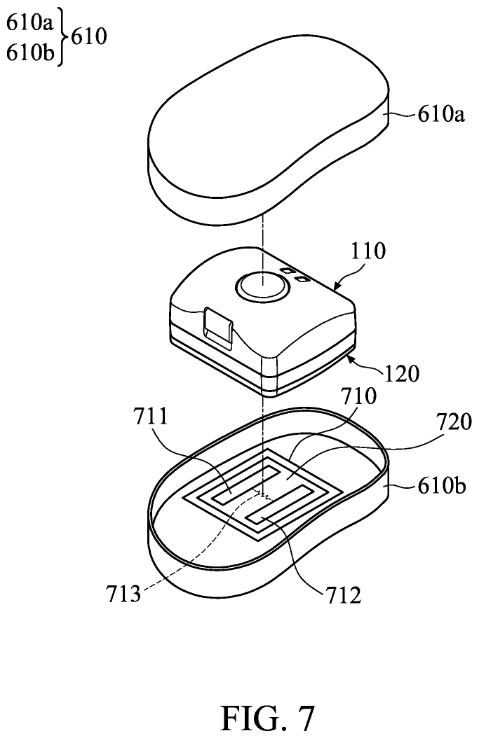

FIG. 7 is a perspective view of a non-implantable electrical stimulation device and a storage box according to an embodiment of the disclosure. Please refer to FIG. 7. The non-implantable electrical stimulation device 700 includes an electrical stimulator 110, an electrode assembly 120 and a storage box 610. In the embodiment, the electrical stimulator 110, the electrode assembly 120 and the storage box 610 in FIG. 0.7 are the same as or similar to the electrical stimulator 110, the electrode assembly 120 and the storage box 610 in FIG. 6A. Accordingly, the electrical stimulator 110, the electrode assembly 120 and the storage box 610 in FIG. 7 may refer to the description of the embodiment of FIG. 6A, and the description thereof is not repeated herein.

In the embodiment, the lower cover 610b may include a circuit board 710 and an isolation component 720. That is, the circuit board 710 may form an area to be treated (i.e., a conductive area) that simulates the skin of the user. The circuit board 710 is disposed on the lower cover 610b, i.e., the circuit board 710 is disposed on a side of the lower cover 610b adjacent to the electrode assembly 120. The circuit board 710 includes a first metal trace 711, a second metal trace 712 and an impedance component 713. The impedance component 713 is connected between the first metal trace 711 and the second metal trace 712. The isolation component 720 is disposed on the circuit board 710 and exposes the first metal trace 711 and the second metal trace 712.

In embodiment, the size of the isolation component 720 is, for example, smaller than the size of the circuit board 710, and the positions of the first metal trace 711 and the second metal trace 712 may correspond to the position of the conductive gel 125 (the first sub conductive gel 125a and the second sub conductive gel 125b), but the embodiment of the disclosure is not limited thereto. In some embodiments, the size of the isolation component 720 may also be, for example, larger than the size of the circuit board 710. In the embodiment, the impedance component 713 is, for example, a resistor, and the isolation component 720 is, for example, a release paper, but the embodiment of the disclosure is not repeated herein.

Furthermore, the storage unit 260 of the electrical stimulator 110 may store the impedance value of the electrical stimulator 110 the impedance value of the electrode assembly 120 excluding the conductive gel 125 and the impedance value of the impedance component 713. In addition, the impedance value of the impedance component 713 may be preset and stored in the storage unit 260. In the embodiment, the impedance value of the impedance component 713 is, for example, 500 ohms, but the embodiment of the disclosure is not limited thereto.

In an operation of the non-implantable electrical stimulation device 700, the electrical stimulator 110 and the electrode assembly 120 are placed in the conductive area (for example, the electrode assembly 120 has been attached to the first metal trace 711 and the second metal trace 712 of the lower cover 610b), and the electrical stimulator 110 is turned on but the course of providing electrical stimulation has not yet started. The control unit 240 controls the electrical stimulation signal generating circuit 220 to generate a first measuring signal. The first measuring signal flows through the conductive area (i.e., the first metal trace 711 and the impedance component 713) through the first electrical connector 113a, the second electrical connector 124a, the conductive component 122 and the conductive gel 125, so as to generate a first signal to be tested.

Then, the control unit 240 receives the above first signal to be tested, for example, through the second metal trace 712, the conductive gel 125, the conductive component 122, the second electrical connector 124b and the first electrical connector 113b, and the first signal to be tested includes a voltage value and a current value. Afterward, the control unit 240 obtains the first total impedance value according to the above first signal to be tested. Then, the control unit 240 obtains the impedance value of the conductive gel 125 according to the above first total impedance value, so as to determine the useful lifespan of the conductive gel 125.

For example, after the control unit 240 obtains the first total impedance value, the control unit 240 may deduct the impedance value of the electrical stimulator 110, the impedance value of the electrode assembly 120 excluding the conductive gel 125 and the impedance value of the impedance component 713 from the first total impedance, so as to obtain the impedance value of the conductive gel 125 and determine the useful lifespan of the conductive gel 125. Furthermore, since the impedance value of the electrical stimulator 110 and the impedance value of the electrode assembly 120 excluding the conductive gel 125 are extremely small (such as about 10-20 ohms), the control unit 240 may ignore the impedance value of the electrical stimulator 110 and the impedance value of the electrode assembly 120 excluding the conductive gel 125. That is, the control unit 240 may deduct the impedance value of the impedance component 713 from the first total impedance value, so as to obtain the impedance value of the conductive gel 125 and determine the useful lifespan of the conductive gel 125. The manner of determining the useful lifespan of the conductive gel 125 is as described above, and the description thereof is not repeated herein.

Then, the manner in which the control unit 240 generates the warning signal according to the impedance value of the conductive gel 125 and the predetermined impedance value may refer to the description of the above embodiment, and the description thereof is not repeated herein. Therefore, the user may place the non-implantable electrical stimulation device 700 on the lower cover 610b and make the electrode assembly 125 to contact the first metal trace 711 and the second metal trace 712, so as to perform the operation for measuring the impedance value of the conductive gel 125 as a basis of replacing the conductive gel 125 or the electrode assembly 120 and increase the convenience of use.

In FIG. 7, the non-implantable electrical stimulation device 700 includes the electrical stimulator 110 and the electrode assembly 120, but the embodiment of the disclosure is not limited thereto. In some embodiments, the non-implantable electrical stimulation device 700 may include an electrical stimulator 410 and an electrode assembly 420. The related operations of the electrical stimulator 410 and the electrode assembly 420 are the same as or similar to the related operations of the electrical stimulator 110 and the electrode assembly 120. Accordingly, the related operations of the electrical stimulator 410 and the electrode assembly 420 may refer to the description of the above embodiment, and the description thereof is not repeated herein.

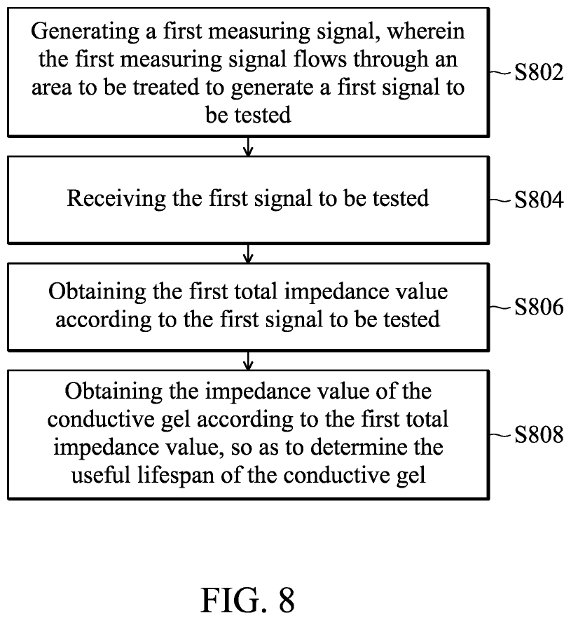

FIG. 8 is a flowchart of an evaluating method for a useful lifespan of a conductive gel according to an embodiment of the disclosure. In the embodiment, the evaluating method for the useful lifespan of the conductive gel is applied to the non-implantable electrical stimulation device 100. The non-implantable electrical stimulation device 100 includes an electrical stimulator 110 and an electrode assembly 120. The electrical stimulator 110 is detachably electrically connected to the electrode assembly 120. The non-implantable electrical stimulation device 100 (i.e., the electrical stimulator 110 and the electrode assembly 120) is placed in the area to be treated (i.e., the conductive area). For example, the electrode assembly 120 has been attached to the area to be treated (i.e., the conductive area). In the embodiment, the area to be treated (i.e., the conductive area) is, for example, the skin of the user.

In step S802, the method involves generating a first measuring signal, wherein the first measuring signal flows through an area to be treated (i.e., the conductive area) to generate a first signal to be tested. In step S804, the method involves receiving the first signal to be tested. In step S806, the method involves obtaining the first total impedance value according to the first signal to be tested. In step S808, the method involves obtaining the impedance value of the conductive gel 125 according to the first total impedance value, so as to determine the useful lifespan of the conductive gel 125.

In some embodiments, the electrical stimulator 110 further stores the impedance value of the electrical stimulator 110, the impedance value of the electrode assembly 120 excluding the conductive gel 125 and the predetermined tissue impedance value. Step S808 further deduct the impedance value of the electrical stimulator 110, the impedance value of the electrode assembly 120 excluding the conductive gel 125 and the predetermined tissue impedance value from the first total impedance value, so as to obtain the impedance value of the conductive gel 125 and determine the useful lifespan of the conductive gel. In some embodiments, since the impedance value of the electrical stimulator 110 and the impedance value of the electrode assembly 120 excluding the conductive gel 125 are extremely small, step S808 may further ignore the impedance value of the electrical stimulator 110 and the impedance value of the electrode assembly 120 excluding the conductive gel 125, and the impedance value of the conductive gel 125 is the first total impedance value deducting the predetermined tissue impedance value.

In some embodiments, the electrical stimulator 110 further stores the impedance value of the electrical stimulator 110 and the impedance value of the electrode assembly 120 excluding the conductive gel 125. Step S808 may further deduct the impedance value of the electrical stimulator 110 and impedance value of the electrode assembly 120 excluding the conductive gel 125 from the first total impedance value, so as to obtain the sum of the impedance value of the conductive gel 125 and the tissue impedance value and determine the useful lifespan of the conductive gel 125 according to the sum of the impedance value of the conductive gel 125 and the tissue impedance value. In some embodiments, since the impedance value of the electrical stimulator 110 and the impedance value of the electrode assembly 120 excluding the conductive gel 125 are extremely small, step S808 may further ignore the impedance value of the electrical stimulator 110 and the impedance value of the electrode assembly 120 excluding the conductive gel 125, and the sum of the impedance value of the conductive gel 125 and the tissue impedance value is the first total impedance value.

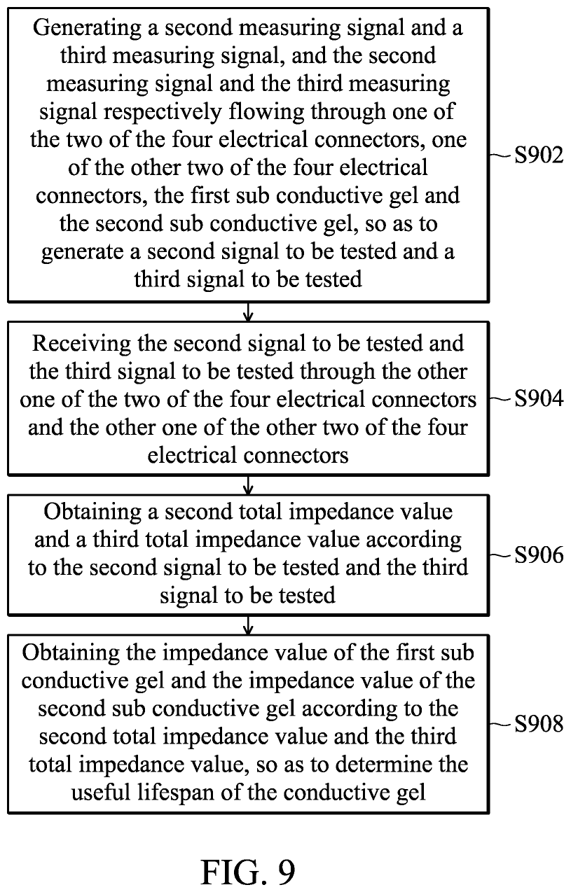

FIG. 9 is a flowchart of an evaluating method for a useful lifespan of a conductive gel according to an embodiment of the disclosure. In the embodiment, the evaluating method for the useful lifespan of the conductive gel is applied to the non-implantable electrical stimulation device 400. The non-implantable electrical stimulation device 400 includes an electrical stimulator 410 and an electrode assembly 420. The electrical stimulator 410 is detachably electrically connected to the electrode assembly 420. The conductive gel 125 of the electrode assembly 420 includes a first sub conductive gel 125a and a second sub conductive gel 125b. In addition, the electrode assembly 420 further includes a conductive component 122 and four electrical connectors (such as the second electrical connectors 124a, 124b, 420a and 420b), wherein the conductive component 122 includes a first sub conductive component 122a and a second sub conductive component 122b, two of the four electrical connectors (such as the second electrical connectors 124a and 420a) penetrate through the first sub conductive component 122a and are electrically connected to the first sub conductive gel 125a, and the other two of the electrical connectors (such as the second electrical connectors 124b and 420b) penetrate through the second sub conductive component 122b and are electrically connected to the second sub conductive gel 125b.

In step S902, the method involves generating a second measuring signal and a third measuring signal, and the second measuring signal and the third measuring signal respectively flowing through one of the two of the four electrical connectors (such as the second electrical connector 124a), one of the other two of the four electrical connectors (such as the second electrical connector 124b), the first sub conductive gel 125a and the second sub conductive gel 125b, so as to generate a second signal to be tested and a third signal to be tested. In step S904, the method involves receiving the second signal to be tested and the third signal to be tested through the other one of the two of the four electrical connectors (such as the second electrical connector 420a) and the other one of the other two of the four electrical connectors (such as the second electrical connector 420b). In step S906, the method involves obtaining a second total impedance value and a third total impedance value according to the second signal to be tested and the third signal to be tested. In step S908, the method involves obtaining the impedance value of the first sub conductive gel 125a and the impedance value of the second sub conductive gel 125b according to the second total impedance value and the third total impedance value, so as to determine the useful lifespan of the conductive gel 125.

In some embodiments, each of the first sub conductive component 122a and the second sub conductive component 122b includes a first portion 520 and a second portion 530 that are separated, one of the two of the four electrical connectors (such as the second electrical connector 124a) penetrates through the first portion 520 of the first sub conductive component 122a and is electrically connected to the first sub conductive gel 125a, the other one of the two of the four electrical connectors (such as the second electrical connector 420a) penetrates through the second portion 530 of the first sub conductive component 122a and is electrically connected to the first sub conductive gel 125a, one of the other two of the four electrical connectors (such as the second electrical connector 124b) penetrates through the first portion 520 of the second sub conductive component 122b and is electrically connected to the second sub conductive gel 125b, and the other one of the other two of the four electrical connectors (such as the second electrical connector 420b) penetrates through the second portion 530 of the second sub conductive component 122b and is electrically connected to the second sub conductive gel 125b.

In some embodiments, the electrical stimulator 410 further stores the impedance value of the electrical stimulator 410 and the impedance value of the electrode assembly 420 excluding the conductive gel 125. Step S908 may further deduct the impedance value of the electrical stimulator 410 and impedance value of the electrode assembly 420 excluding the conductive gel 125 from the second total impedance value, so as to obtain the impedance value of the first sub conductive gel 125a, and deduct the impedance value of the electrical stimulator 410 and impedance value of the electrode assembly 420 excluding the conductive gel 125 from the third total impedance value, so as to obtain the impedance value of the second sub conductive gel 125b, thereby determining the useful lifespan of the conductive gel 125. In the embodiments, since the impedance value of the electrical stimulator 410 and the impedance value of the electrode assembly 420 excluding the conductive gel 125 are extremely small, step S908 may further ignore the impedance value of the electrical stimulator 410 and the impedance value of the electrode assembly 420 excluding the conductive gel 125, and the impedance value of the first sub conductive gel 125a is the second total impedance value and the impedance value of the second sub conductive gel 125b is the third total impedance value.

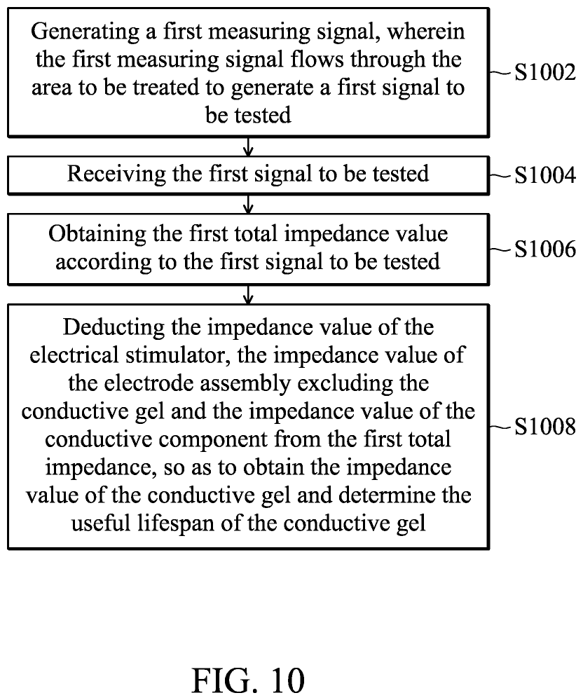

FIG. 10 is a flowchart of an evaluating method for a useful lifespan of a conductive gel according to an embodiment of the disclosure. In the embodiment, the evaluating method for the useful lifespan of the conductive gel is applied to the non-implantable electrical stimulation device 600. The non-implantable electrical stimulation device 600 includes an electrical stimulator 110, an electrode assembly 120 and a storage box 610. The electrical stimulator 110 is detachably electrically connected to the electrode assembly 120. The storage box 610 includes an upper cover 610a and a lower cover 610b. The upper cover 610a and the lower cover 610b form an accommodating space, and the accommodating space is configured to accommodate the electrical stimulator 110 and the electrode assembly 120.

The lower cover 610b includes a conductive component 620 and an isolation component 630. The area to be treated (i.e., the conductive area) may be the conductive component 620. The conductive component 620 is disposed on the lower cover 610b, and the isolation component 630 is disposed on a part of the conductive component 620. The electrical stimulator 110 and the electrode assembly 120 are placed in the area to be treated (i.e., the conductive area). For example, the electrode assembly 120 has been attached to the conductive component 620 of the lower cover 610b. The electrical stimulator 110 further stores the impedance value of the electrical stimulator 110, the impedance value of the electrode assembly 120 excluding the conductive gel 125 and the impedance value of the conductive component 620.

In step S1002, the method involves generating a first measuring signal, wherein the first measuring signal flows through the area to be treated (i.e., the conductive area) to generate a first signal to be tested. In step S1004, the method involves receiving the first signal to be tested. In step S1006, the method involves obtaining the first total impedance value according to the first signal to be tested. In step S1008, the method involves deducting the impedance value of the electrical stimulator 110, the impedance value of the electrode assembly 120 excluding the conductive gel 125 and the impedance value of the conductive component 620 from the first total impedance, so as to obtain the impedance value of the conductive gel 125 and determine the useful lifespan of the conductive gel 125.

In some embodiments, since the impedance value of the electrical stimulator 110 and the impedance value of the electrode assembly 120 excluding the conductive gel 125 are extremely small, step S1008 may further ignore the impedance value of the electrical stimulator 110 and the impedance value of the electrode assembly 120 excluding the conductive gel 125, and the impedance value of the conductive gel 125 is the first total impedance value deducting the impedance value of the conductive component 620.

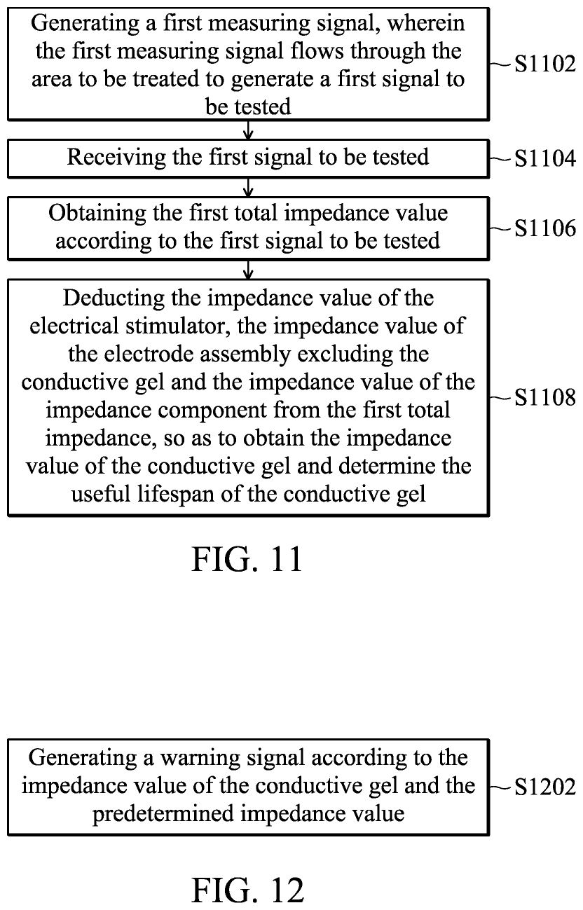

FIG. 11 is a flowchart of an evaluating method for a useful lifespan of a conductive gel according to an embodiment of the disclosure. In the embodiment, the evaluating method for the useful lifespan of the conductive gel is applied to the non-implantable electrical stimulation device 700. The non-implantable electrical stimulation device 700 includes an electrical stimulator 110, an electrode assembly 120 and a storage box 610. The electrical stimulator 110 is detachably electrically connected to the electrode assembly 120. The storage box 610 includes an upper cover 610a and a lower cover 610b. The upper cover 610a and the lower cover 610b form an accommodating space, and the accommodating space is configured to accommodate the electrical stimulator 110 and the electrode assembly 120.

The lower cover 610b includes a circuit board 710 and an isolation component 720. The area to be tested (i.e., the conductive area) may be the circuit board 710. The circuit board 710 is disposed on the lower cover 610b. The circuit board 710 includes a first metal trace 711, a second metal trace 712 and an impedance component 713. The impedance component 713 is connected between the first metal trace 711 and the second metal trace 712. The isolation component 720 is disposed on the circuit board 710 and exposes the first metal trace 711 and the second metal trace 712. The electrical stimulator 110 and the electrode assembly 120 are placed in the area to be treated (i.e., the conductive area). For example, the electrode assembly 120 has been attached to the first metal trace 711 and the second metal trace 712 of the lower cover 610b. The electrical stimulator 110 further stores the impedance value of the electrical stimulator 110, the impedance value of the electrode assembly 120 excluding the conductive gel 125 and the impedance value of the impedance component 713.

In step S1102, the method involves generating a first measuring signal, wherein the first measuring signal flows through the area to be treated (i.e., the conductive area) to generate a first signal to be tested. In step S1104, the method involves receiving the first signal to be tested. In step S1106, the method involves obtaining the first total impedance value according to the first signal to be tested. In step S1108, the method involves deducting the impedance value of the electrical stimulator 110, the impedance value of the electrode assembly 120 excluding the conductive gel 125 and the impedance value of the impedance component 713 from the first total impedance, so as to obtain the impedance value of the conductive gel 125 and determine the useful lifespan of the conductive gel 125.

In some embodiments, since the impedance value of the electrical stimulator 110 and the impedance value of the electrode assembly 120 excluding the conductive gel 125 are extremely small, step S1108 may further ignore the impedance value of the electrical stimulator 110 and the impedance value of the electrode assembly 120 excluding the conductive gel 125, and the impedance value of the conductive gel 125 is the first total impedance value deducting the impedance value of the impedance component 713.

FIG. 12 is a flowchart following step S808 of FIG. 8, step S908 of FIG. 9, step S1008 of FIG. 10 and step S1108 of FIG. 11. In step S1202, the method involves generating a warning signal according to the impedance value of the conductive gel 125 and the predetermined impedance value.

In summary, according to the evaluating method for the useful lifespan of the conductive gel and the non-implantable electrical stimulation device disclosed by the present disclosure, the first measuring signal is generated, and the first measuring signal flows through the area to be treated (i.e., the conductive area) to generate the first signal to be tested. The first total impedance value is obtained according to the first signal to be tested. The impedance value of the conductive gel is obtained according to the first total impedance value, so as to determine the useful lifespan of the conductive gel. In addition, the embodiment may generate the warning signal according to the impedance value of the conductive gel and the predetermined impedance value. Therefore, the impedance value of the conductive gel may be effectively obtained, and the useful lifespan (state) of the conductive gel may be determined according to the impedance value of the conductive gel, so as to decide whether to replace the conductive gel or the electrode assembly, and the convenience of use is increased.

In addition, the conductive gel of the embodiment may include the first sub conductive gel and the second sub conductive gel, the embodiment may generate the second measuring signal and the third measuring signal, and the second measuring signal and the third measuring signal respectively flow through the first sub conductive gel and the second conductive gel, so as to generate the second signal to be tested and the third signal to be tested. The second total impedance value and the third total impedance value are obtained according to the second signal to be tested and the third signal to be tested. The impedance value of the first sub conductive gel and the impedance value of the second sub conductive gel are obtained according to the second total impedance value and the third total impedance value. Therefore, when the non-implantable electrical stimulation device is not attached to the area to be tested, the impedance value of the conductive gel may be measured and obtained to determine the useful lifespan of the conductive gel, so as to increase the convenience of use.

Furthermore, the non-implantable electrical stimulation device of the embodiment further includes the storage box. The component (such as the conductive component or the circuit board) corresponding to the area to be tested (i.e., the conductive area) is disposed on the lower cover of the storage unit. The electrical stimulator and the electrode assembly are placed on the lower cover, and the impedance value of the conductive gel may be measured and obtained, so as to determine the useful lifespan of the conductive gel. Therefore, the convenience of us may also be increased.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An evaluating method for a useful lifespan of a conductive gel, applied to a non-implantable electrical stimulation device, wherein the non-implantable electrical stimulation device comprises an electrical stimulator and an electrode assembly, the electrical stimulator is detachably electrically connected to the electrode assembly, and the evaluating method for the useful lifespan of the conductive gel comprises:
generating a first measuring signal, wherein the first measuring signal flows through a conductive area to generate a first signal to be tested;
receiving the first signal to be tested;
obtaining a first total impedance value according to the first signal to be tested; and
obtaining the impedance value of the conductive gel according to the first total impedance value, so as to determine a useful lifespan of the conductive gel.

2. The evaluating method for the useful lifespan of the conductive gel as claimed in claim 1, wherein the step of obtaining the impedance value of the conductive gel according to the first total impedance value, so as to determine the useful lifespan of the conductive gel, comprises:
deducting an impedance value of the electrical stimulator, an impedance value of the electrode assembly excluding the conductive gel and a predetermined tissue impedance value from the first total impedance value, so as to obtain the impedance value of the conductive gel and determine the useful lifespan of the conductive gel.

3. The evaluating method for the useful lifespan of the conductive gel as claimed in claim 2, wherein the impedance value of the electrical stimulator and the impedance value of the electrode assembly excluding the conductive gel are ignored, and the impedance value of the conductive gel is the first total impedance value deducting the predetermined tissue impedance value.

4. The evaluating method for the useful lifespan of the conductive gel as claimed in claim 2, wherein when the impedance value of the conductive gel is greater than or equal to 600 to 800 ohms, it is determined that the useful lifespan of the conductive gel is unusable.

5. The evaluating method for the useful lifespan of the conductive gel as claimed in claim 1, wherein the step of obtaining the impedance value of the conductive gel according to the first total impedance value, so as to determine the useful lifespan of the conductive gel, comprises:
deducting an impedance value of the electrical stimulator and an impedance value of the electrode assembly excluding the conductive gel from the first total impedance value, so as to obtain a sum of the impedance value of the conductive gel and a tissue impedance value and determine the useful lifespan of the conductive gel according to the sum of the impedance value of the conductive gel and the tissue impedance value.

6. The evaluating method for the useful lifespan of the conductive gel as claimed in claim 5, wherein the impedance value of the electrical stimulator and the impedance value of the electrode assembly excluding the conductive gel are ignored, and the sum of the impedance value of the conductive gel and the tissue impedance value is the first total impedance value.

7. The evaluating method for the useful lifespan of the conductive gel as claimed in claim 5, wherein when the sum of the impedance value of the conductive gel and the tissue impedance value is greater than or equal to 1000 to 2000 ohms, it is determined that the useful lifespan of the conductive gel is unusable.

8. The evaluating method for the useful lifespan of the conductive gel as claimed in claim 1, wherein the non-implantable electrical stimulation device further comprises a storage box, the storage box comprises an upper cover and a lower cover, the upper cover and the lower cover form an accommodating space, and the accommodating space is configured to accommodate the electrical stimulator and the electrode assembly;
wherein the lower cover comprises a conductive component and an isolation component, wherein the conductive area is the conductive component, the conductive component is disposed on the lower cover, and the isolation component is disposed on a part of the conductive component;
wherein the step of obtaining the impedance value of the conductive gel according to the first total impedance value, so as to determine the useful lifespan of the conductive gel, comprises:
deducting an impedance value of the electrical stimulator, an impedance value of the electrode assembly excluding the conductive gel and an impedance value of the conductive component from the first total impedance, so as to obtain the impedance value of the conductive gel and determine the useful lifespan of the conductive gel.

9. The evaluating method for the useful lifespan of the conductive gel as claimed in claim 8, wherein the impedance value of the electrical stimulator and the impedance value of the electrode assembly excluding the conductive gel are ignored, and the impedance value of the conductive gel is the first total impedance value deducting the impedance value of the conductive component.

10. The evaluating method for the useful lifespan of the conductive gel as claimed in claim 1, wherein the non-implantable electrical stimulation device further comprises a storage box, the storage box comprises an upper cover and a lower cover, the upper cover and the lower cover form an accommodating space, and the accommodating space is configured to accommodate the electrical stimulator and the electrode assembly;
wherein the lower cover comprises a circuit board and an isolation component, wherein the conductive area is the circuit board, the circuit board is disposed on the lower cover, the circuit board comprises a first metal trace, a second metal trace and an impedance component, the impedance component is connected between the first metal trace and the second metal trace, and the isolation component is disposed on the circuit board and exposes the first metal trace and the second metal trace;
wherein the step of obtaining the impedance value of the conductive gel according to the first total impedance value, so as to determine the useful lifespan of the conductive gel, comprises:
deducting an impedance value of the electrical stimulator, an impedance value of the electrode assembly excluding the conductive gel and an impedance value of the impedance component from the first total impedance, so as to obtain the impedance value of the conductive gel and determine the useful lifespan of the conductive gel.

11. The evaluating method for the useful lifespan of the conductive gel as claimed in claim 10, wherein the impedance value of the electrical stimulator and the impedance value of the electrode assembly excluding the conductive gel are ignored, and the impedance value of the conductive gel is the first total impedance value deducting the impedance value of the impedance component.

12. A non-implantable electrical stimulation device, comprising:
an electrode assembly, comprising a conductive gel; and
an electrical stimulator, wherein the electrical stimulator is detachably electrically connected to the electrode assembly and the electrical stimulator comprises:
an electrical stimulation signal generating circuit, configured to generate a first measuring signal, and the first measuring signal flows through a conductive area to generate a first signal to be tested; and
a control unit, configured to receive the first signal to be tested, obtain a first total impedance value according to the first signal to be tested, and obtain the impedance value of the conductive gel according to the first total impedance value, so as to evaluate a useful lifespan of the conductive gel.

13. The non-implantable electrical stimulation device as claimed in claim 12, wherein the electrical stimulator further comprises a storage unit for storing an impedance value of the electrical stimulator, an impedance value of the electrode assembly excluding the conductive gel and a predetermined tissue impedance value;
wherein the control unit deducts the impedance value of the electrical stimulator, the impedance value of the electrode assembly excluding the conductive gel and the predetermined tissue impedance value from the first total impedance value, so as to obtain the impedance value of the conductive gel and determine the useful lifespan of the conductive gel.

14. The non-implantable electrical stimulation device as claimed in claim 12, wherein the electrical stimulator further comprises a storage unit for storing an impedance value of the electrical stimulator and an impedance value of the electrode assembly excluding the conductive gel;
wherein the control unit deducts the impedance value of the electrical stimulator and the impedance value of the electrode assembly excluding the conductive gel from the first total impedance value, so as to obtain a sum of the impedance value of the conductive gel and a tissue impedance value, and determine the useful lifespan of the conductive gel according to the sum of the impedance value of the conductive gel and the tissue impedance value.

15. The non-implantable electrical stimulation device as claimed in claim 12, wherein the conductive gel comprises a first sub conductive gel and a second sub conductive gel;
wherein the electrode assembly further comprises a conductive component and four electrical connectors, wherein the conductive component comprises a first sub conductive component and a second sub conductive component, two of the four electrical connectors penetrate through the first sub conductive component and are electrically connected to the first sub conductive gel, and the other two of the electrical connectors penetrate through the second sub conductive component and are electrically connected to the second sub conductive gel;
wherein the electrical stimulation signal generating circuit further generate a second measuring signal and a third measuring signal, and the second measuring signal and the third measuring signal respectively flow through one of the two of the four electrical connectors, one of the other two of the four electrical connectors, the first sub conductive gel and the second sub conductive gel, so as to generate a second signal to be tested and a third signal to be tested;
wherein the control unit further respectively receives the second signal to be tested and the third signal to be tested through the other one of the two of the four electrical connectors and the other one of the other two of the four electrical connectors, obtains a second total impedance value and a third total impedance value according to the second signal to be tested and the third signal to be tested, and obtains an impedance value of the first sub conductive gel and an impedance value of the second sub conductive gel according to the second total impedance value and the third total impedance value.

16. The non-implantable electrical stimulation device as claimed in claim 15, wherein each of the first sub conductive component and the second sub conductive component includes a first portion and a second portion that are separated, the one of the two of the four electrical connectors penetrates through the first portion of the first sub conductive component and is electrically connected to the first sub conductive gel, the other one of the two of the four electrical connectors penetrates through the second portion of the first sub conductive component and is electrically connected to the first sub conductive gel, the one of the other two of the four electrical connectors penetrates through the first portion of the second sub conductive component and is electrically connected to the second sub conductive gel, and the other one of the other two of the four electrical connectors penetrates through the second portion of the second sub conductive component and is electrically connected to the second sub conductive gel.

17. The non-implantable electrical stimulation device as claimed in claim 15, wherein the electrical stimulator further comprises a storage unit for storing an impedance value of the electrical stimulator and an impedance value of the electrode assembly excluding the conductive gel;
wherein the control unit deducts the impedance value of the electrical stimulator and the impedance value of the electrode assembly excluding the conductive gel from the second total impedance value, so as to obtain the impedance value of the first sub conductive gel, and deducts the impedance value of the electrical stimulator and the impedance value of the electrode assembly excluding the conductive gel from the third total impedance value, so as to obtain the impedance value of the second sub conductive gel.

18. The non-implantable electrical stimulation device as claimed in claim 12, further comprising a storage box, wherein the storage box comprises an upper cover and a lower cover, the upper cover and the lower cover form an accommodating space, and the accommodating space is configured to accommodate the electrical stimulator and the electrode assembly;
wherein the lower cover comprises a conductive component and an isolation component, wherein the conductive area is the conductive component, the conductive component is disposed on the lower cover, and the isolation component is disposed on a part of the conductive component;
wherein the electrical stimulator further comprises a storage unit for storing an impedance value of the electrical stimulator, an impedance value of the electrode assembly excluding the conductive gel and an impedance value of the conductive component;
wherein the control unit deducts the impedance value of the electrical stimulator, the impedance value of the electrode assembly excluding the conductive gel and the impedance value of the conductive component from the first total impedance value, so as to obtain the impedance value of the conductive gel and determine the useful lifespan of the conductive gel.

19. The non-implantable electrical stimulation device as claimed in claim 12, further comprising a storage box, wherein the storage box comprises an upper cover and a lower cover, the upper cover and the lower cover form an accommodating space, and the accommodating space is configured to accommodate the electrical stimulator and the electrode assembly;
wherein the lower cover comprises a circuit board and an isolation component, wherein the conductive area is the circuit board, the circuit board is disposed on the lower cover, the circuit board comprises a first metal trace, a second metal trace and an impedance component, the impedance component is connected between the first metal trace and the second metal trace, and the isolation component is disposed on the circuit board and exposes the first metal trace and the second metal trace;
wherein the electrical stimulator further comprises a storage unit for storing an impedance value of the electrical stimulator, an impedance value of the electrode assembly excluding the conductive gel and an impedance value of the impedance component;
wherein the control unit deducts the impedance value of the electrical stimulator, the impedance value of the electrode assembly excluding the conductive gel and the impedance value of the impedance component from the first total impedance value, so as to obtain the impedance value of the conductive gel and determine the useful lifespan of the conductive gel.

20. The non-implantable electrical stimulation device as claimed in claim 12, wherein the electrical stimulator further comprises a warning unit;
wherein the control unit is further configured to generate a warning signal to the warning unit according to the impedance value of the conductive gel and a predetermined impedance value, so that the warning unit displays the warning signal.
